(19) United States
(12) Patent Application Publication
(10) Pub. No.: US 2006/0128699 A1
(43) Pub. Date: Jun. 15, 2006

(54) Novel pharmacophore for the discovery and testing of na,k-atpase inhibitor compositions and methods for their use in treating cardiovascular diseases and conditions

(76) Inventors: William J Welsh, Princeton, NJ (US); Susan M Keenan, Levittown, PA (US); Robert K Delisle, Levittown, PA (US); William J Ball, Cincinnati, OH (US)

Correspondence Address: Licata & Tyrrell, 66 East Main Street, Marlton, NJ, 08053, US

(21) Appl. No.: 10/534,296

(22) PCT Filed: Nov. 7, 2003
(86) PCT No.: PCT/US03/35636

§ 371 Date: Dec. 9, 2005

Related U.S. Application Data
(60) Provisional application No. 60/425,037, filed on Nov. 7, 2002.

(51) Int. Cl. A61K 31/538; A61K 31/38; A61K 31/353; C40B 30/02; C40B 40/02; G06F 19/00

(52) U.S. Cl. 514/229.8; 514/431; 514/454; 435/7.1; 702/19

(57) ABSTRACT

Disclosed is a novel pharmacophore and novel inotropic compositions created from the novel pharmacophore. Also disclosed are methods of making and using the novel pharmacophore and method of use of novel compositions to treat heart diseases by inhibiting Na, K-ATPase.

### CROSS REFERENCE TO RELATED APPLICATIONS

This application is a continuation-in-part of U.S. Provisional Application Ser. No. 60/425,037, filed Nov. 7, 2002.

### FIELD OF THE INVENTION

The present invention relates to the discovery of a three dimensional pharmacophore based on a model of the Na, K-ATPase that will enable the discovery and design of novel positive inotropic agents using molecular modeling, pharmacophores, rational drug design techniques, and methods of using the novel compounds in treating heart diseases.

### BACKGROUND OF THE INVENTION

Each of the references cited herein is incorporated by reference in its entirety.

Heart diseases, such as cardiac failure, paroxysmal atrial tachycardia, and caridomyopathy, are a major cause of morbidity and mortality around the world. Cardiac failure occurs when the heart becomes unable to pump blood effectively at a rate that meets the needs of the metabolizing tissues. The underlying cause of cardiac failure lies in the reduced contractility of heart muscles. Reduced contraction of the heart leads to reduced heart output of blood volume while the amount of blood being returned to the heart remains the same. The result is an increase in heart blood volume, which in turn leads to heart congestion. Congestive heart failure causes lowered blood pressure, poor renal blood flow with concomitant edema in the lower extremities and pulmonary edema, as well as renal failure. Positive inotropic activity, meaning increasing the force of the contraction of the heart, is thus a primary goal in the treatment of heart failure patients.

While recent advances in diagnostic and surgical procedures have revolutionized cardiovascular medicine, similar progress has not been attained with respect to pharmacological agents. Increasing the force of contraction of the failing heart is still largely achieved by cardioglycosides (also called cardioactive glycosides) derived from the plant Digitalis purpurea, (foxglove) such as digoxin and digitoxin. Unfortunately, digoxin, digitoxin, and other cardioglycoside drugs, the most widely used clinical inotropic agents, have narrow therapeutic indices and a high potential for considerable toxic effects including ventricular arrhythmias. Therefore, the difference between the dosage leading to a therapeutic increase in heart muscle contraction and the dosage leading to fatal toxicity is minute. This potentially deadly property severely curtails the clinical usefulness of the currently available drugs. While much effort has been directed toward the discovery of novel therapeutic agents, the lack of three-dimensional structural coordinates for the receptor of inotropic drugs, Sodium Potassium (Na, K) ATPase, has markedly limited success. Increasing the therapeutic index of positive inotropic Na, K-ATPase (also referred to as Na+, K+-ATPase herein) inhibitors would thus represent a major advance in the clinical management of heart disease.

Sodium Potassium ATPase is a heterodimeric transmembrane protein that actively exchanges sodium and potassium ions across cell membranes using energy from the hydrolysis of ATP. Inhibition of the Na, K-ATPase in the myocardial cells of the heart muscle results in the sequential increase of cytosolic sodium and calcium concentrations, thereby increasing the force of contraction. Regulation of the levels of sodium and potassium contribute to many essential physiological processes including maintenance of the membrane potential for muscle contraction.

Cardioglycoside inhibitors bind to the extracellular domain of the catalytic 1α subunit of the Na, K-ATPase and inhibition of the ATPase results in both the beneficial and detrimental effects associated with cardioglycoside administration. While researchers have obtained low resolution crystal structures (˜6 Å) of the Na, K-ATPase and gained structural insights from solid-state NMR studies of ATPase-inhibitor binding, the dearth of detailed structural information has hindered the discovery of novel inotropic inhibitors. One of the main obstacles to Na, K-ATPase crystallization is that the protein is structurally complex, possessing ten transmembrane spanning domains (H1-H10), which makes the development of the high quality crystals of integral membrane proteins required for X-ray crystallography exceedingly difficult.

The exact mechanism of inhibition has remained elusive. Although researchers have examined alternative inhibitory agents, of the hundreds of cardioglycosides tested, none have been shown to have larger therapeutic indices than existing therapeutic agents do. Moreover the lack of structural knowledge of the Na, K-ATPase has thwarted the structural investigation and structure based discovery of novel alternative treatments. A novel approach to the design, discovery, and use of inotropic drugs with reduced toxicity would thus be very useful in the treatment of heart failure.

### SUMMARY OF THE INVENTION

The present invention creates a novel pharmacophore from which to generate scaffolds, and then molecules, which have inotropic properties. Preferably, the pharmacophore is defined by the parameters of Table 4 and Table 5, which reference the spheres in FIG. 4. The invention also contemplates the method of creating the novel pharmacophore.

Another aspect of the present invention sets forth a basic scaffold created using the novel pharmacophore. The basic scaffold molecule is a novel Na, K-ATPase ligand. The scaffold can be used to develop positive inotropic drugs that inhibit Na, K-ATPase activity.

A third aspect of the present invention discloses further developed potential scaffolds of the novel Na, K-ATPase ligands. These scaffolds represent the parent molecules, which satisfy the basic three point pharmacophores. The Na, K-ATPase inhibitor compounds are of the formula: 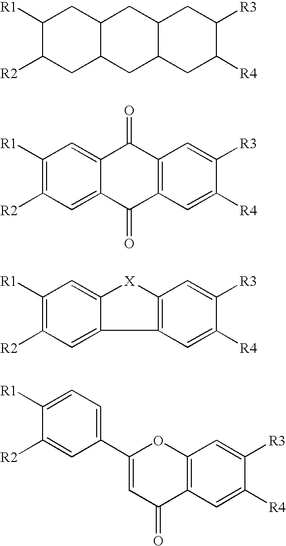
wherein R1, R2, R3 and R4 can be any organic functional group containing a hydrogen bond donor or a hydrogen bond acceptor and X is any element or group that allows the compound to retain inotropic activity. Preferably, X is N, O, S, or C.

A further aspect of the present invention takes the scaffold molecules and develops them into functional drugs for Na, K-ATPase inhibition, which can be used to prevent or treat heart conditions, particularly congestive heart failure and supraventricular arrhythmia. Eight compounds have shown promise as inotropic agents, with three of the eight demonstrating high therapeutic potential based on their ATPase inhibitory characteristics.

These and other aspects of the invention will be further elucidated in the following descriptions and examples. The descriptions in the present invention are provided only as examples and should not be understood to be limiting on the claims. Based on the description, a person of ordinary skill in the art may make modifications and changes to the preferred embodiments, which does not depart from the scope of the present invention.

### BRIEF DESCRIPTION OF THE DRAWINGS

FIG. 1. This figure shows how the threaded alignment of the 1α-subunit of SERCA1a (skeletal muscle sarcoplasmic reticulum/endoplasmic reticulum Ca₂⁺-ATPase) [SEQ ID NO: 1] with the sheep Na, K-ATPase [SEQ ID NO: 2] was accomplished. The alignment shown is the result of “threading” the Na⁺, K⁺-ATPase sequence to that of the template. Identical residues between the two sequences are illustrated with filled black boxes, while similar residues correspond to the filled gray boxes. Regions modeled are noted by lines above lettered designations, with the letter shown at the beginning and end of the sequence encoding the region. The letters correspond to letters attached to the helices and loops depicted in FIGS. 2A and 2B. The solid lines under the sequence of FIG. 1 represent helical structures, while the broken lines are indication of loop regions. Local areas of high identity correlating with functional motifs are shown by open black boxes and described in the text. Positions of sequence changes between the sheep and rat Na⁺, K⁺-ATPases are denoted with an asterisk (*).

FIG. 2. This figure shows the resulting homology model of the extracellular loops and the transmembrane domains regions of the sheep 1α-subunit of the Na, K-ATPase. Shown is a visual representation of the three-dimensional coordinates assigned to the homology model (2A), and a representation of the α-helices from the extracellular domain (2B). Only alpha carbons are depicted. The numbering, showing the orientation of the helices, is consistent with the information obtained from low-resolution structures. Tryptophan and lysine residues are displayed (2A). The lettering corresponds to the amino acid sequence portions shown in FIG. 1.

FIG. 3. This figure shows the binding orientation of cardioglycosides to the 1α subunit of the Na, K-ATPase predicted by the methods of the invention. White numbers in the figure indicate transmembrane domains. The extracellular (3A) and cross-section (3B) represent the consensus orientation for all nine docked cardioglycosides. Both Na⁺, K⁺-ATPase and ligands are depicted with Connelly surfaces. The figure depicts consensus orientation of 25 individual dockings of ouabain to the 1α-subunit of the sheep Na⁺, K⁺-ATPase (3C). Orientation of digoxin (3D, upper) and digoxigenin bisdigitoxose (3D, lower) illustrate the identical conformation regardless of the number of sugar moieties.

FIG. 4. This figure shows the pharmacophoric elements fitting the binding criteria for 1α-subunit Na, K-ATPase inhibition. Each pharmacophoric element as described herein is represented by a point encased in a sphere. The spheres (diameter=2.5 Å) represent the tolerance of location allowed during database searching. The pharmacophoric elements are combined to form three, four and five point pharmacophores representing increasing stringency of search criteria (Table 4).

### DETAILED DESCRIPTION OF THE INVENTION

### DEFINITIONS

The present invention can best be understood in light of the following definitions.

“Inotropic” means affecting the force or energy of muscular contractions.

“Glycosides” are any compound that contains a constituent sugar in which the hydroxyl group attached to the first carbon is substituted with an alcoholic, phenolic, or other group.

“Rational drug design” means modeling the molecular structure of the target of a drug and then designing a drug that will attack the target.

“Pharmacophore” is a model for developing one or more molecular scaffolds or structure used as the basis for drug development.

“Scaffold” means a supporting framework, such as the basic backbone or structure of a molecule with designated positions capable of containing a one or more molecules or functional groups.

“Dynamic programming” is a use of scoring matrices and gap penalties to produce optimal alignment.

“Unaltered” means not changed, substituted, deleted, or mutated.

“Pharmaceutically effective dose” as used herein refers to the amount of an novel inotropic compound or novel compound composition comprising one or more of the novel compounds described herein that produces a desired therapeutic effect, such as treating the target disease. The precise amount of the pharmaceutically effective dose of a novel compound or novel compound composition that will yield the most effective results in terms of efficacy of treatment in a given subject will depend upon the activity, pharmacokinetics, pharmacodynamics, and bioavailability of a particular inotropic compound, physiological condition of the subject, including age, gender, disease type and stage, general physical condition, responsiveness to a given dosage and type of medication, the nature of pharmaceutically acceptable carrier in a formulation, and a route of administration, among other potential factors. Those skilled in the clinical and pharmacological arts will be able to determine these factors through routine experimentation consisting of monitoring the subject and adjusting the dosage. Remington: The Science and Practice of Pharmacy (Gennaro ed. 20^{th} edition, Williams & Wilkins PA, USA) (2000).

As used herein, the “target disease” may be any disease in which inhibiting Na, K-ATPase treats the disease. Without limitation, the target diseases are heart diseases, particularly congestive heart failure and supraventricular arrhythmia. While it is possible for a novel compound to be administered as a pure or substantially pure compound, it is preferable that the novel inotropic compound be administered as a composition in the form of pharmaceutical formulations or preparations suitable for a particular administration route. A novel compound composition comprises one or more novel inotropic compounds and a pharmaceutically acceptable carrier.

The term “pharmaceutically acceptable carrier” as used herein means a pharmaceutically-acceptable material, composition or vehicle, such as a liquid or solid filler, diluent, excipient, solvent or encapsulating material, involved in carrying or transporting one or more novel inotropic compounds from one tissue, organ, or portion of the body, to another tissue, organ, or portion of the body. The novel inotropic compounds may be administered in liposomes. Each component must be “pharmaceutically acceptable” by being compatible with the other ingredients of the formulation. It must also be suitable for use in contact with the tissue or organ of humans and animals, specifically the heart and circulatory system, without excessive toxicity, irritation, allergic response, immunogenecity, or other problems or complications, commensurate with a reasonable benefit/risk ratio.

A “route of administration” for a novel compound or composition can be by any pathway known in the art, including without limitation, oral, enteral, nasal, topical, rectal, vaginal, aerosol, transmucosal, transdermal, ophthalmic, pulmonary, and/or parenteral administration. A parenteral administration refers to an administration route that typically relates to injection. Parenteral administration includes, but is not limited to, intravenous, intramuscular, intraarterial, intraathecal, intracapsular, infraorbital, intracardiac, intradermal, intraperitoneal, transtracheal, subcutaneous, subcuticular, intraarticular, subcapsular, subarachnoid, intraspinal, via intrasternal injection, and/or via infusion.

“Treatment” of or “treating” a disease may mean preventing the disease by causing clinical symptoms not to develop, inhibiting the disease by stopping or reducing the symptoms, the development of the disease, and/or slowing the rate of development of the disease, relieving the disease by causing a complete or partial regression of the disease, reducing the risk of developing the disease, or a combination thereof.

The term “contacted” when applied to a cell, tissue or organ means the process by which a novel compound or compound composition is delivered to the target cell, tissue or organ, or placed in direct proximity of the cell, tissue, or organ.

“Therapeutically effective amount” is the amount of novel compound or composition that, when administered to a subject, is effective to bring about a desired effect. In this case, that effect is typically an antagonistic effect that ultimately decreases the activity of the Na, K-ATPase.

“Pharmaceutically acceptable carriers” for the novel inotropic compositions may include sugars, starches, cellulose, powdered tragacanth; malt; gelatin; talc; excipients, oils, glycols, esters, agar; buffering agents, and other non-toxic compatible substances employed in pharmaceutical formulations. The formulations may conveniently be presented in unit dosage form and may be prepared by any methods well known in the pharmaceutical arts. Formulations suitable for oral administration may be in the form of capsules, cachets, pills, tablets, lozenges, powders, as granules, bolus, electuary, or a paste, as a solution or a suspension in an aqueous or non-aqueous liquid, as an oil-in-water or water-in-oil liquid emulsion, as an elixir or syrup, or as pastilles, each containing a predetermined amount of one or more of the novel inotropic compositions as an active ingredient.

In solid forms for oral administration, capsules, tablets, pills, powders, granules and the like, may by used. In the case of capsules, tablets and pills, the pharmaceutical compositions may also comprise buffering agents. Compressed tablets may be prepared using binder, lubricant, inert diluent, preservative, or disintegrant. Liquid dosage forms for oral administration include pharmaceutically acceptable emulsions, microemulsions, solutions, suspensions, syrups and elixirs. Formulations for the topical or transdermal administration of a novel inotropic composition include powders, sprays, ointments, pastes, creams, lotions, gels, solutions, patches and inhalants, such as by aerosol.

Formulations suitable for parenteral administration comprise a novel inotropic composition in combination with one or more pharmaceutically acceptable sterile isotonic aqueous or nonaqueous solutions, dispersions, suspensions or emulsions, or sterile powders which may be reconstituted into sterile injectable solutions or dispersions. The formulations may contain antioxidants, buffers, bacterostats, solutes that render the formulation isotonic with the blood of the intended recipient or suspending or thickening agents. Formulations suitable for parenteral administration may also contain adjuvants such as preservatives, wetting agents, emulsifying agents and dispersing agents, various antibacterial and antifungal agents, or agents that delay absorption such as aluminum monostearate and gelatin.

Aspects

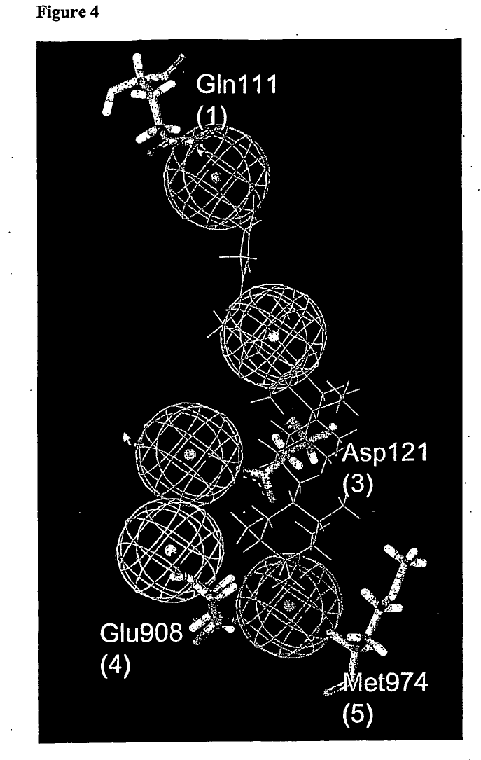

A novel pharmacophore is disclosed. The pharmacophore allows the generation of basic scaffold molecules, which can then be transformed into more detailed scaffold molecules having inotropic properties. The pharmacophore is created to generate Na, K-ATPase ligands and is defined by the parameters of Table 4 and Table 5 and the spheres in FIG. 4. The more detailed scaffolds can then be used to create potential drugs, which can also be tested within the pharmacophore model for binding potential. The scaffolds at least satisfy the basic three point pharmacophores, but may satisfy the four or five point pharmacophores.

The Na, K-ATPase inhibitor scaffold compounds may be of the formula: 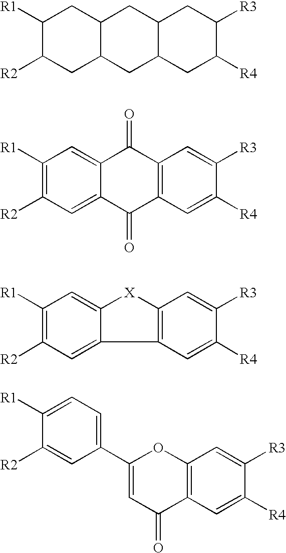
for example, wherein R1, R2, R3 and R4 can be any organic functional group containing a hydrogen bond donor or a hydrogen bond acceptor and X is any element or group that allows the compound to retain inotropic activity. Preferably, X is a single element, such as N, O, S, or C.

The scaffold molecules are the basis functional drugs for Na, K-ATPase inhibition, which can be used to prevent or treat heart conditions, particularly congestive heart failure and supraventricular arrhythmia. Eight compounds have been tested shown as inotropic agents, with three of the eight drugs have demonstrated particularly high potential. These drugs are shown below:
(1) (+)-Catechin Hydrate (experimentally noted as Compound 9) 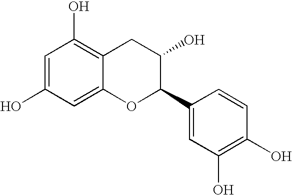
(2) 4′-5′ Di nitroflorescein (experimentally noted as Compound 14). This compound demonstrates strong ATPase inhibition. 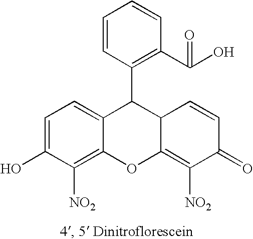
(3) Emodin (experimentally noted as Compound 15). 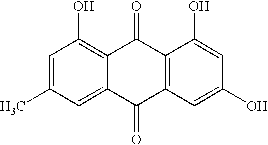
(4) Lacmoid (experimentally noted as Compound 19). This compound demonstrates strong ATPase inhibition. 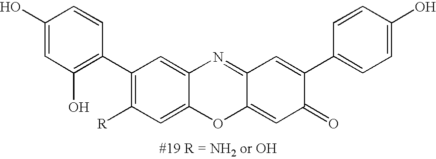
(5) R—S Dobutamine (experimentally noted as Compound 25). 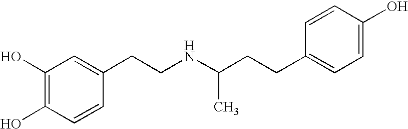
(6) Compound 26 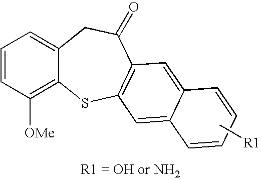
(7) Compound 27 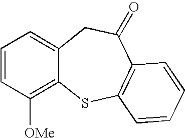
(8) Compound 28. This compound demonstrates strong ATPase inhibition.

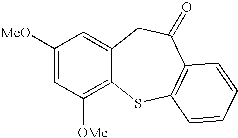

The data for the tests of the eight compounds is found in Tables 1a and 1b.

TABLE 1a
Activity of NKA assay started by addition of ATP
units are nM

| compound ID | IC50_1 | IC50_2 | IC50_3 | IC50_4 | IC50_5 | IC50_aver. | ratio (digoxin) |
|---|---|---|---|---|---|---|---|
| digoxin | 478 | 337 | — | — | — | 407 | 1 |
| ouabain | 498 | 518 | 523 | 628 | 440 | 521 | 1.28 |
| 14 | 5689 | 8867 | 11707 | — | | 8754 | 21.5 |
| 19 | 11246 | 192410 | 9703 | — | | 13386 | 32.9 |
| 28 | 358753 | — | — | — | | 358753 | 881 |
| 9 | 287309 | 252885 | — | — | | 270097 | 664 |
| 25 | 64424 | 76751 | — | — | | 70587 | 173 |
| 26 | 216847 | 291204 | — | — | | 254025 | 624 |
| 27 | 385505 | 102282 | 119777 | — | | 202521 | 497 |
| 28 | 35517 | 42480 | 36316 | 144801 (?) | | 38104 | 94 |
| 15 | 32202 | 75482 | 161602 | — | | 89762 | 220 |

TABLE 1b
Activity of NKA assay started by addition of KCl
Cardoglycosides will show greater affinity with KCl added
last rather than ATP

| compound | | | | | |
|---|---|---|---|---|---|
| ID | IC50_1 | IC50_2 | IC50_3 | IC50_aver. | ratio (digoxin) |
| digoxin | 4.6 | 15 | 12 | 10 | 1 |
| ouabain | 4 | 12 | 10 | 9 | 0.9 |
| 14 | 7806 | 9528 | — | 8692 | 869 |
| 19 | 3554 | 3246 | — | 3400 | 340 |
| 28 | 75572 | 59710 | — | 67641 | 6764 |

The drugs found to be efficacious in treating heart diseases will be administered to individuals in need thereof in pharmaceutically effective amounts for the duration of the disease, until the disease has abated, or as needed to prevent the disease. The novel inotropic compounds should also be administered in a pharmaceutically acceptable carrier. There are many methods and routes of administration and possible carriers, which are discussed in detail in the definitions section. An ordinarily skilled clinician will be able to determine the appropriate amount of the one or more novel inotropic compounds to administer to an individual. Preferably, the novel inotropic compound has a wider therapeutic index that the commonly used cardioglycoside drugs, such as dioxin or ouabain.

Finally, a general method of making and using the pharmacophore to create an Na, K-ATPase inhibitory compound comprises:
(a) creating alignment between SERCA1a and Na, K-ATPase, wherein SERCA1a acts as the structural template, and the alignment comprises use of both dynamic programming and threading techniques;
(b) transferring coordinates from a template to a model for structurally conserved regions;
(c) generating variable regions;
(d) refining the model through energy minimization steps; and
(e) performing docking analysis of prospective drug candidates.
   The process may further comprise the steps of:
(f) delineating the essential pharmacophoric elements for high binding affinity;
(g) searching databases of known compounds using the restraints as implicated by the pharmacophore with allowable tolerances (e.g. ±10%); and
(h) utilizing de novo rational drug design and computer aided molecular modeling to design novel compounds using the restraints as implicated by the pharmacophore with allowable tolerances (e.g. ±10%).

As now explained in further detail, the present invention has found a way to utilize the recent solution of the structural coordinates of the E₁ conformation of SERCA1a (skeletal muscle sarcoplasmic reticulum/endoplasmic reticulum Ca₂⁺-ATPase) as a structural template for homology three-dimensional modeling of the Na, K-ATPase. SERCA also occurs in the E2 conformation, and in SERCA1b, and SERCA2 forms. The Na, K-ATPase and other P₂-type ATPases are thought to share important structural features with SERCA1a and sequence analysis of the family members established that these structures are evolutionarily related.

Accordingly, by the methods of the instant invention, a model of the extracellular and transmembrane domains of the human 1α-subunit of the Na, K-ATPase has been developed, starting from the high-resolution crystal structure of SERCA1a. The first and most important step in any modeling process is the alignment of the protein sequence to be modeled to that of the structural template. With a sequence identity of only ˜25%, aligning the 1α-subunit of the Na, K-ATPase to SERCA1a by virtue of residue identity/homology is thwarted with complications and thus such an alignment cannot be confidently used as a basis for a three-dimensional structure.

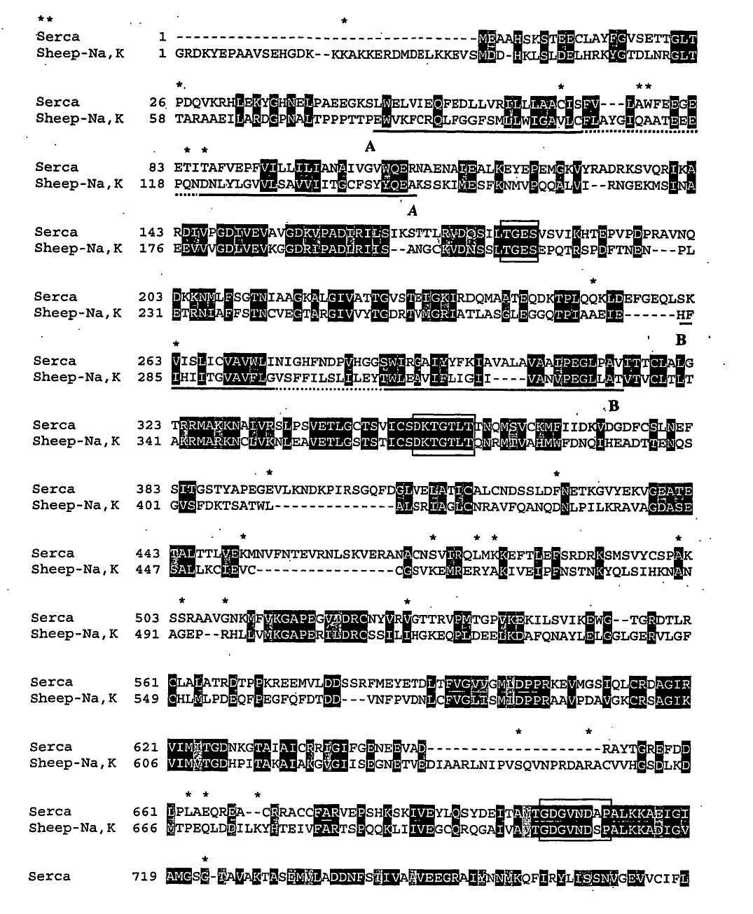

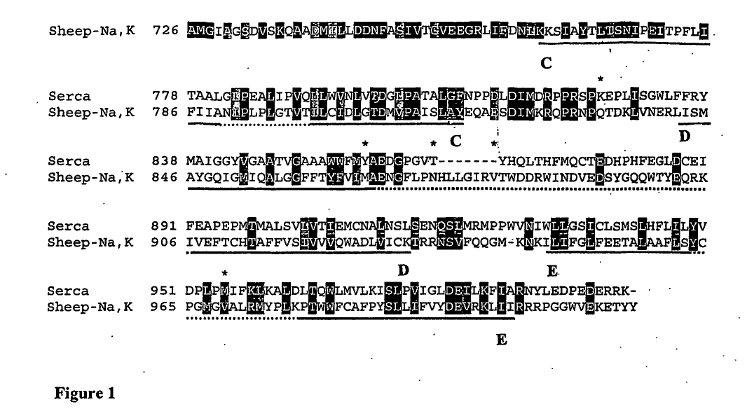

Therefore, in one preferred embodiment, the invention takes advantage of the known structural and functional similarities of the family of P₂-Type ATPases and uses a technique that essentially “threads” the sequence of interest to libraries of known protein folds from resolved structures. In this manner, the possible tertiary structure configurations that provide environments that satisfy the primary sequence of the 1α-subunits of the Na, K-ATPase can be investigated. Through the independent alignment of the sheep 1α-subunit sequences to SERCA1a, the instant invention has achieved alignments that have a high level of confidence. Areas of high local sequence similarity (indicated by shaded black and gray boxes, FIG. 1) are well aligned as exemplified by the perfect alignment of the 7 residues (DKTTGTLT) (residues 351-357 of SEQ ID NO:1) that comprise the E1-E2 ATPases (aspartate)phosphorylation site that is ubiquitous to P₂-type ATPases. The TGES (residues 181-184 of SEQ ID NO:1) sequence prior to the phosphorylation site and the GDGXNDXP (where X is any residue) (residues 701-708 of SEQ ID NO:1) motif (FIG. 1, open black boxes) are further evidence of the alignment. These data demonstrate that after aligning the sequences without bias to residue identity, the resulting orientation effectively aligns these essential regions.

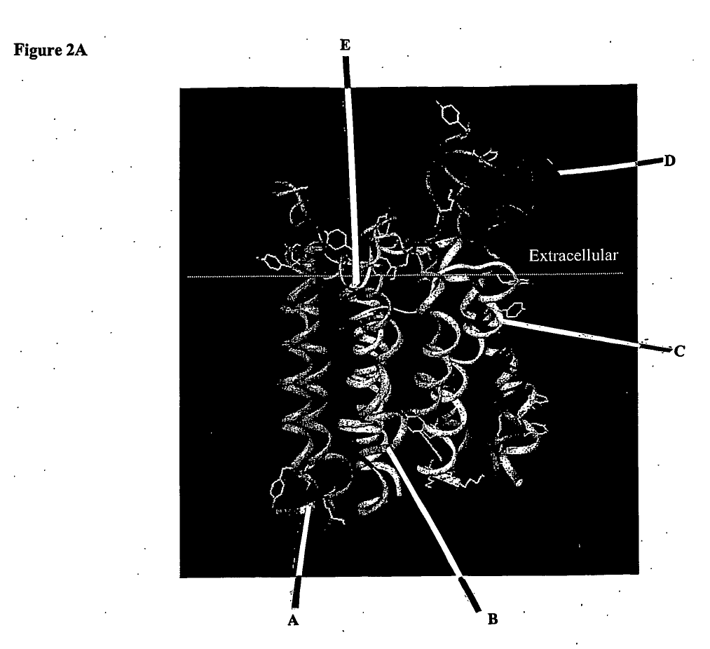

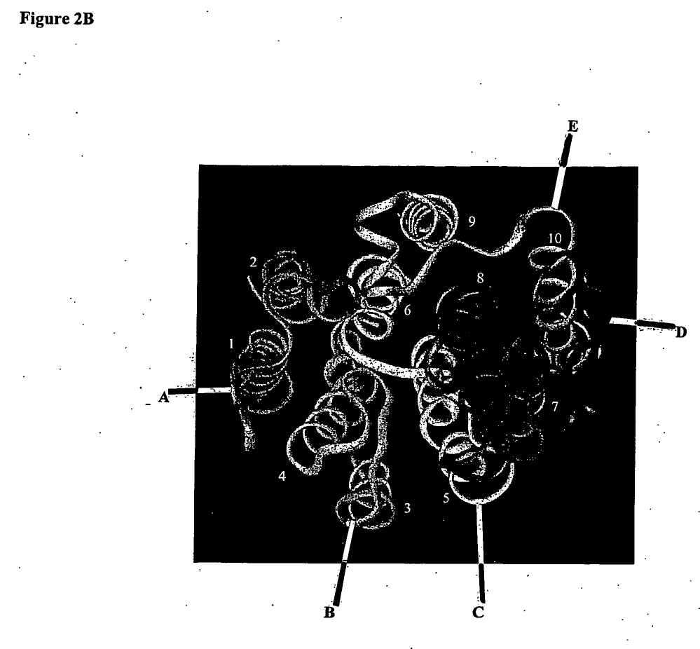

Further extrapolating from the structural conservation of P₂-type ATPases, the present invention uses computational homology modeling techniques to derive a three-dimensional structure for the extracellular and transmembrane domains of the 1α subunits of both human and sheep Na, K-ATPases. Thus, in another preferred embodiment of the invention, these three-dimensional models and specific mutations within the extracellular domain as they relate to ouabain affinity/binding are applied to examine the mode of inhibitor-receptor association. As ouabain and the other cardioglycosides bind to the extracellular domain of the 1α-subunit, five structurally conserved regions (SCRs) are modeled which collectively consist of the ten transmembrane domains and connecting extracellular loop regions. These SCRs are notated by solid numbered lines in FIG. 1 and the numbers correlate with the helices depicted in FIG. 2. Areas with extended regions of hydrophobicity in the primary sequence are then compared to the transmembranes predicted by the alignment, those predicted by secondary structure predictors, and the transmembrane domains proposed as a result of hydrophobicity studies. The results of the analyses are tabulated (Table 2) and the high level of consistency provide additional evidence to corroborate the alignment scheme of the invention. Furthermore, when a standard pairwise alignment is performed using a hydrophobicity matrix, the fundamental alignment (FIG. 1) is maintained. Consequently, supportive data from three independent methods as well as structural motif conservation are obtained, all of which converge on the final sequence alignment of the present invention.

TABLE 2

| | | Hydrophobicity | |
|---|---|---|---|
| Helices | Alignment | Plots | Memsat |
| 1 | 83-109 | 89-110 | 89-110 |
| 2 | 126-149 | 123-140 | 123-142 |
| 3 | 285-298 | 282-304 | 287-305 |
| 4 | 316-337 | 311-342 | 314-338 |
| 5 | 768-789 | 770-791 | 771-791 |
| 6 | 799-819 | 796-818 | 800-817 |
| 7 | 845-871 | 841-865 | 842-866 |
| 8 | 909-934 | 909-930 | 914-930 |
| 9 | 948-969 | 945-970 | 946-964 |
| 10 | 981-997 | 975-996 | 978-994 |

In another preferred embodiment of the invention, the structural coordinates of the helical regions of sheep 1α-subunit are assigned, and loops are generated that are sterically acceptable and that result in reasonable phi and psi angles for the spliced regions (InsightII, Accelrys). The resulting structure is then subjected to a series of refinement steps. Initially, the entire protein is relaxed and thermodynamically energy minimized to reduce initial steric bumps resulting from residue replacement within the secondary structural features. Next, the residue side chains are checked for appropriate rotomer positioning using a standard rotomer library defined from known crystal structures. Finally, the model is energy minimized through a series of steps in which hydrogens, side chains and finally, the entire molecule are successively allowed freedom of movement. Ribbon representations of the cross-section and the extracellular side views of the final sheep model are shown in FIGS. 2A and 2B respectively.

The resulting model can be structurally validated by the use of the wealth of biological data available for the sheep 1α-subunit for structural validation. Site directed chemical labeling of the extracellular loops of the sheep Na, K-ATPase has been utilized to suggest structural orientation. Cysteine residues for P118, T309, L793, L876, and M973 have previously been introduced to investigate membrane topology. All of the introduced cysteine residues could be chemically labeled indicating that these residues are exposed to the extracellular surface and our model places all five residues in extracellular loop regions. Studies have shown that the preference of tryptophan residues and to a lesser extent lysine residues for membrane surfaces is a feature ubiquitous to membrane proteins. Analysis of the positioning of these residues in the model of the present invention shows that in fact these amino acids do tend to associate close to the proposed transmembrane surfaces, providing further validation of the model. Moreover, as expected, there appears to be a propensity for tryptophan residues to be localized on the extracellular surface while lysine residues appear to be incorporated on the intracellular membrane. In summary, both the alignment and model of the present invention are strongly supported by experimental data.

In another preferred embodiment, with the information obtained from the three dimensional structures of the transporter, the invention provides a pharmacophore model for the Na, K-ATPase, which describes the types of atoms or pharmacophoric elements and their geometric arrangements that are common across the set of active cardioglycosides in relation to the constraints imposed by the molecular binding site. The three-dimensional model of the pharmacophore of invention makes it possible for the first time to examine the molecular mechanism of Na, K-ATPase inhibition, to screen for novel inotropic inhibitors of the Na, K-ATPase with higher therapeutic indices, as well as to design such inhibitors through the use of rational drug design techniques. With the 3D-model of the Na, K-ATPase provided by the present invention, the mode of cardioglycoside binding can now be elucidated. Three-dimensional quantitative structural-activity relationship models (QSAR) based on the inhibition of the sheep 1α-subunit as a result of incubation with cardioactive compounds previously developed and the resulting comparative molecular field analysis (CoMFA) models provide structural implications for the binding site based on the correlation of changes in ligand conformation and biological activity. In combination with the model of the present invention, these models suggest that the length of the binding pocket is ˜20 Å, which is corroborated by earlier estimates of ˜19 Å. Mutational studies suggest that the ligand binding site is situated between H1-H2 (Ribbon No. 1, FIG. 2A) and H5-H6 (Ribbon No. 3, FIG. 2A), and H1-H2 and H9-H10 (Ribbon No. 5, FIG. 2A) corresponding to distances of 20 Å and 22 Å respectively. Collectively these data suggest that the binding site consists of a ˜20 Å groove comprised of the extracellular loops connecting H1-H2, H5-H6, and H9-H10.

Thus, in another preferred embodiment, the present invention provides a three-dimensional model of the Na, K-ATPase cardiotonic binding site. In a further preferred embodiment of the invention, a pharmacophore model is provided, which precisely defines the mechanism of interaction between the Na, K-ATPase and an inhibitory molecule. This model allows for the de novo design of drug candidates based on the Na, K-ATPase receptor structure. The pharmacophore allows for the development of inotropic agents that are structurally unique from those chemical classes known and examined to date, thus making it possible to develop inotropic agents with higher (also called wider) therapeutic indices.

The systematic docking of 16 cardioglycosides inside the putative Na, K-ATPase binding pocket provided by the instant invention was achieved by using ) GOLD (Genetic Optimization for Ligand Docking) Jones et al. J. Mol. Biol 254, 43-53 (1995). However, any accurate docking program is contemplated, including conformational sampling-based methods (e.g., free energy perturbation, linear interaction energy approximations), knowledge-based “potential of mean force” methods (Pmf, Drugscore), force-field methods (GOLD, Dock, AutoDock), Apropros, CombiBUILD, Escher, FlexiDock, FLOG, GRAMM, HEX, HotDock, LIGIN, PUZZLE, STALK, and empirical free-energy scoring functions (Ludi, Chemscore, Score, Fresno, FlexX, Plp). In addition, the docking and scoring methods of U.S. Provisional Application No. 60/477,714, entitled “Novel Knowledge-based Docking and Scoring Algorithm for Virtual Drug Screening (Welsh, et al.), filed Jun. 10, 2003 is incorporated by reference herein.

GOLD is a ligand-docking program, which uses a genetic algorithm (GA) to explore ligand conformation and satisfy ligand-binding requirements (http://www.ccdc.cam.ac.uk/prods/gold). One advantage over other docking systems is that GOLD allows not only for ligand flexibility, but also for limited flexibility of the binding pocket creating a more realistic environment for inhibitor association. Further, the stochastic nature of GOLD ensures that the search space is well explored and local extremes are less influential as compared to deterministic algorithms. Thus, it was possible to select a subset of inhibitors consisting of all molecules 15-30 Å in length and including ouabain, digoxin and digitoxin. These molecules best fit the binding site of the invention, and as a result are unlikely to have more than one mode of association. The combination of experimental and computational results suggests that the ligand binding domain is ˜20 Å which effectively defines a docking sphere of a ˜10 Å radius. A centroid of the sphere was determined as the intersection of the vectors defining H1-H2 with H5-H6, H1-H2 with H9-H10 and H5-H6 with H9-H10. Each inhibitor was docked 25 independent times.

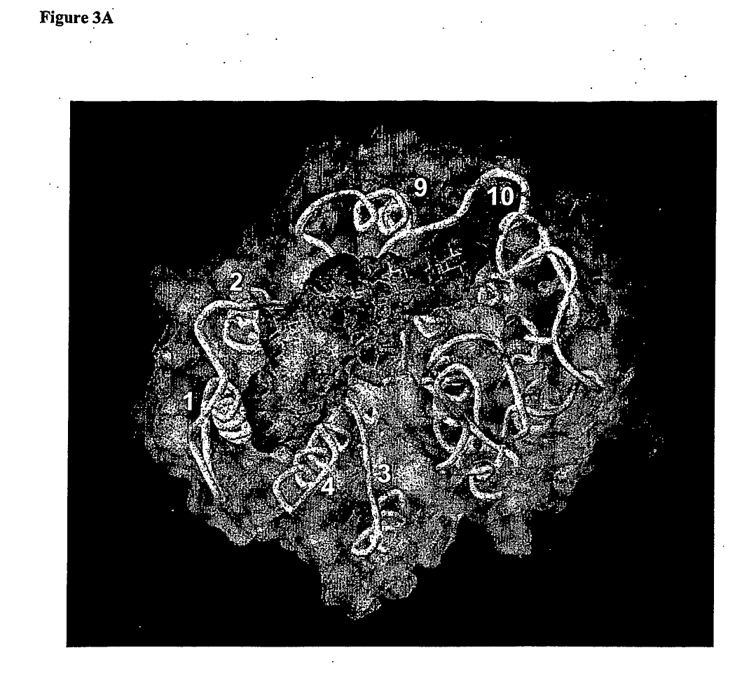

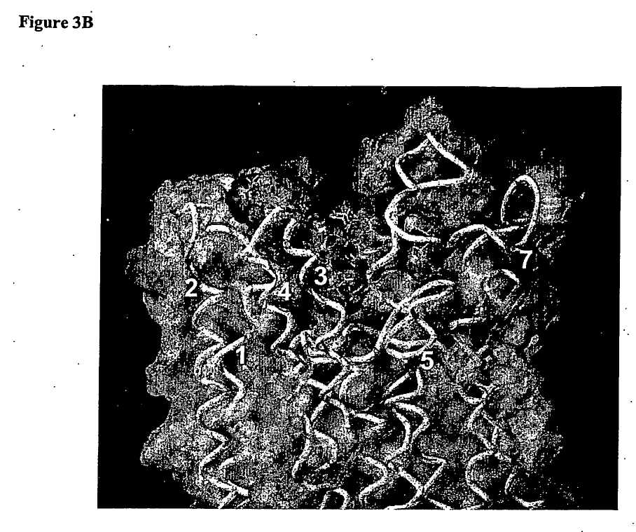

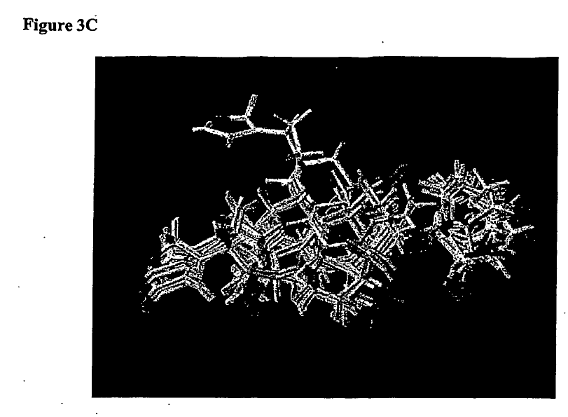

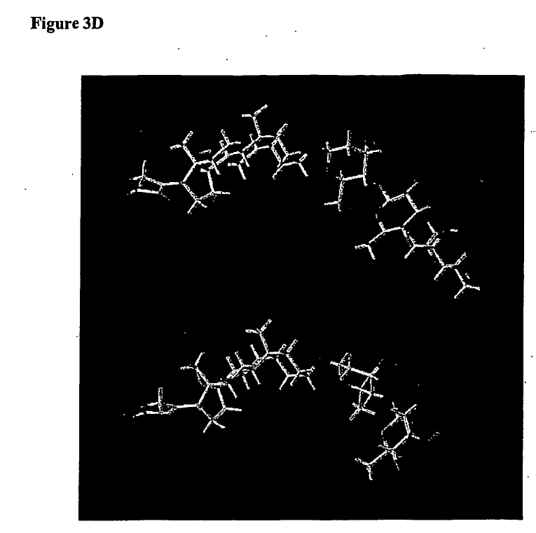

To explore more thoroughly the protein-inhibitor interaction, the radii of the docking spheres were varied (with increasing radii 10 Å, 12 Å, and 15 Å). Similar results were obtained with the two smaller docking spheres while the excessively large volume of the 15 Å docking sphere resulted in unrealistic interactions in which the molecules docked perpendicular to the molecular surface of the Na, K-ATPase. Using the results generated with the 12 Å-docking sphere, the consensus mode of binding for each inhibitor from the family of 25 dockings was determined. As all the binding modes were rank ordered using the GOLD fitness function, which measures steric and electrostatic complementarity between each ligand conformation and the receptor, the molecule corresponding to the highest scoring conformation of each binding consensus was selected. As an example, the alignment of the 25 conformations of ouabain (FIG. 3A) and the overall alignment of all 16 inhibitors representing the consensus conformation (FIG. 3B) is provided. It is notable that while GOLD is a stochastic algorithm, the independent dockings converged to a single consensus both within dockings of an independent compound as well as across dockings between compounds. In a manner consistent with experimental findings, the cardiotonic steroid moiety and the unsaturated lactone ring are oriented toward H1-H2, while the carbohydrates are directed toward H9-H10. Compounds with more than one sugar see these moieties wrap around H7-H8. All compounds completely occupy the potential binding pocket which is situated over the channel (depicted by a fast Connolly channel surface) thus effectively inhibiting the function of the ATPase by blocking the ion channel (FIG. 3C).

The model of the binding site allows for two primary observations: First, that the consensus binding orientation is highly conserved. With no outliers, the docking modeling results in a single binding mode and suggests that only the cardiotonic steroid moiety, lactone ring and alpha sugar contribute to binding. Second, while the overall domain of binding is very consistent, the individual orientation of the steroid backbones of the inhibitors is not uniform indicating that this moiety is acting as a scaffold which provides appropriate spacing for the functional groups, rather than as a pharmacophoric element essential to binding. Furthermore, the number of sugars has no effect on the binding consensus. For example, digitoxin with two or three sugars maintains an identical binding orientation (FIG. 3D). This observation agrees not only with previous in vitro studies describing the orientation of the sugars of bound inhibitors, but furthermore corresponds with our own observations based on our CoMFA models.

One factor that influences the Gibb's free energy (ΔG) of binding of a small molecule to a receptor is the formation of hydrogen bonds between the compound and the receptor. One of the most widely used definitions to determine hydrogen bonding potential for secondary structure determination is the Database of Secondary Structure in Proteins or DSSP. Systematic measurement of the distance between all potential hydrogen bond donors and/or acceptors of the ligands and the residues comprising the binding pocket of the invention, the potential residue interactions of each compound within the binding site were mapped. Of the 16 molecules docked to the 1α-subunit, 15 had the potential to form hydrogen bonds with the side chain or backbone nitrogen of residue D121. Biological data has suggested that D121 residue is essential for inhibitor binding as a single point mutation (D121N) at this location decreases sensitivity to ouabain by 1000 fold. Four additional residues also appear to be involved in molecular inhibition: the side chains of Q111 and N122, and the backbone nitrogens of E908 and M973. The data provided by the methods and models of the present invention show that 15 of the 16 molecules docked could potentially form hydrogen bonds with Q111, and mutants of the 1α-subunit of the Na, K-ATPase with R replacing residue Q111, or R and D replacing residues Q111 and N122 respectively, are 8.3 fold and 1150 fold less sensitive to ouabain than the wild-type sheep catalytic subunit. In addition, all 16 molecules were able to form hydrogen bonds with the backbone nitrogen of E908, and 12 molecules were shown to have the potential to interact with M973.

The effects of these mutations on the binding of the cardioglycosides can further be predicted by modeling the 1α-subunit of the Na, K-ATPase incorporating either the Q111R or D121N mutations. In addition, the mutations were introduced in the models of the human and rat 1α-subunits of the Na, K-ATPase whose sequence differs from that of the sheep by 23 residues (or 2%) and 35 residues (3.5%) respectively. The human 1α-subunit remains sensitive to ouabain while the rat subunit is insensitive to ouabain inhibition. The docking study with ouabain was repeated for both the human and rat 1α-subunits, and the mutant structures maintaining identical parameters as previously. Ouabain binding to the human 1α-subunit of the Na, K-ATPase is identical to that observed in the original docking study. The binding affinity of ouabain for both the human and the sheep 1α-subunit of the Na, K-ATPase is analogous, thus inclusion of the human model acts as a control for the following mutational studies. The incorporation of either single point mutation (Q111R or D121N) results in the reorientation of the lactone moiety from H1-H2 toward H3-H4. Furthermore, the rat (which contains both Q111R and N122D point mutations) forces ouabain to abandon the consensus orientation observed with the human and sheep subunits.

The mutational effects with respect to ouabain sensitivity, described above, can be further explored computationally as a decrease in receptor sensitivity to an inhibitor often reflects diminished binding affinity. As such, binding energies (ΔE_{binding}) associated with various ouabain-enzyme interactions resulting from the docking studies were calculated and then compared with available inhibition data. In order to quantify the reorientation of ouabain with respect to the affinity decreases observed experimentally, the relative change in binding energy (ΔE_{binding}) were calculated. Values of ΔE_{binding} were computed as the difference in calculated energy between the ouabain-receptor complex (E_{complex}) and the sum of the energies calculated separately for the free ligand (E_{ligand}) and the free receptor (E_{receptor}).

The molecular mechanics calculations included bonded and nonbonded (vdW and Coulombic) energy terms and the electrostatic component of solvation. The ΔE_{binding} data (Table 3) shows a strong statistical correlation with the experimentally derived inhibitory effects of the mutants (Pearson correlation r=0.81; Spearman correlation r=0.88). The calculated values of ΔE_{binding} for the wild-type sheep and human al-subunits are consistent with their nearly equivalent ouabain affinity. Furthermore, introduction of the Q111R mutation which is known to lower ouabain's affinity for the sheep enzyme by ˜10-fold resulted in a corresponding decrease in the calculated binding affinity. Furthermore, the rat α1-subunit, or introduction of the D121N into the sheep α1 model, yielded ΔE_{binding} values consistent with the dramatic loss (˜1000 fold) loss in ouabain inhibition associated with either of these enzymes.

As the results of our molecular modeling studies have implicated two additional residues as important components of the digitalis binding site, it was sought to further investigate the role of these amino-acid residues with respect to ouabain binding. While in vitro mutation studies are beyond the scope of the present study, these mutations were simulated in silico to determine the influence of these residues on the affinity of ouabain. Mutant sheep α1-subunits were constructed incorporating either an E908L or M973A mutation and docking analyses with ouabain were performed. The consensus orientation of ouabain selected for analysis was obtained as described above.

Interestingly, the consensus orientation observed from the docking ensemble differed from the consensus orientation of ouabain bound to the wild-type sheep enzyme suggesting that these residues each independently play a role in ouabain binding. Values of ΔE_{binding} were calculated (Table 4) and compared with the ΔE_{binding} values calculated for the wild-type and mutant sheep-, rat- and human enzymes. The results indicate that both mutants could have a large impact on ouabain binding affinity.

TABLE 3
Calculated changes in Binding Energies (ΔE_{binding})

| Species/Mutant* | Relative Inhibition | ΔΔE_{binding} (kcal/mol) |
|---|---|---|
| Sheep | 1 | −204 |
| Rat | 1000 | −158 |
| Human | 1 | −199 |
| D121N | 1000 | −107 |
| Q111R | 10 | −167 |
| E908L | ukn | −160 |
| M973A | ukn | −131 |

*Mutation of sheep structure.

In another preferred embodiment, the invention provides a method of identifying novel ligands of the Na, K-ATPase. Identification of novel ligands can be achieved by methods known in the art, such as the screening of libraries and databases for chemicals including proteins, peptides, nucleotides, and small molecules that correspond to the structural criteria defined by the pharmacophore of the invention. The pharmacophore of the present invention contains the essential hydrophobic and hydrophilic regions for interaction (FIG. 4).

There are essentially three regions of the binding pocket that could be essential for binding: a hydrogen bond donor/acceptor corresponding to the potential hydrogen bond donor/acceptor of Q111, an intervening hydrophobic area, and a hydrogen bond donor corresponding to the potential hydrogen bond acceptor D121 (represented respectively by spheres 1,2 and 3; FIG. 4). The two hydrogen bonding groups are 9.95 Å and 7.63 Å from the hydrophobic core, resulting in a total distance of 17.10 Å separating the two hydrogen bonding (FIG. 4A). In addition to the three pharmacophoric elements discussed above, the 3D coordinates of E908 (sphere 4; FIG. 4) and M973 (sphere 5; FIG. 4) provide positioning for auxiliary potential hydrogen bond donor/acceptor groups. The rigor of the database search criteria can thus be increased in a stepwise fashion by introducing four and five point pharmacophores (Table 4).

TABLE 4

| Pharmacophore | Sphere 1 | Sphere 2 | Sphere 3 | Sphere 4 | Sphere5 |
|---|---|---|---|---|---|
| Three Point | HBD | HYD | HBD | | |
| | HBA | HYD | HBD | | |
| Four Point | HBD | HYD | HBD | HBD | |
| | HBA | HYD | HBD | HBA | |
| | HBD | HYD | HBD | | HBD |
| | HBA | HYD | HBD | | HBA |
| | HBD | HYD | HBD | HBA | |
| | HBA | HYD | HBD | HBD | |
| | HBD | HYD | HBD | | HBA |
| | HBA | HYD | HBD | | HBD |
| Five Point | HBD | HYD | HBD | HBD | HBD |
| | HBD | HYD | HBD | HBD | HBA |
| | HBD | HYD | HBD | HBA | HBD |
| | HBD | HYD | HBD | HBA | HBA |
| | HBA | HYD | HBD | HBD | HBD |
| | HBA | HYD | HBD | HBD | HBA |
| | HBA | HYD | HBD | HBA | HBD |
| | HBA | HYD | HBD | HBA | HBA |

*HBA represents Hydrogen Bond Acceptor; HBD represents Hydrogen Bond Donor; HYD represents Hydrophobic group

Distance between points are located in Table 5.

TABLE 5

| Pharmacophoric | | | | |
|---|---|---|---|---|
| Elements | 2 | 3 | 4 | 5 |
| 1 | 15 ± 0.75 | 8 ± 0.40 | 18 ± 0.90 | 22 ± 1.10 |
| 2 | — | 9 ± 0.45 | 6 ± 0.30 | 9 ± 0.45 |
| 3 | — | — | 12 ± 0.60 | 14 ± 0.70 |
| 4 | — | — | — | 7 ± 0.35 |

* All measurements are in Å.

However, compounds do not have to satisfy all criteria to be active in inhibiting the 1αsubunit of the ATPase. The combination of the initial three pharmacophoric elements (spheres 1, 2, and 3) collectively represent the minimum 3D criteria necessary to search existing databases for novel potential scaffolds. To validate the basic three-point pharmacophores of the invention, NCI and Maybridge three-dimensional databases were searched for molecules that satisfy the pharmacophoric parameters. The pharmacophores of the invention allowed the identification of existing cardioglycosides that were independent of those used for pharmacophore development in the databases utilizing the pharmacophore. Therefore, the three-dimensional selection criteria developed by the present invention mirror the physical elements required for inhibitor binding and can be therefore used for the design of new therapeutic agents. In addition to the identification of cardioglycosides, the database search identified several new potential scaffolds of interest. Based on the models and methods of the invention, it was shown that the cardiosteroid backbone of the inhibitors are acting merely as hydrophobic spacers, and therefore substitution of scaffolds that maintain the three dimensional orientation of the pharmacophoric elements should succeed in inhibiting the Na, K-ATPase.

Thus, in yet another preferred embodiment of the invention, a method of designing ligands that will inhibit the Na, K-ATPase is provided. The structural data provided by the models and methods of the present invention make it possible to elucidate the mechanism of cardioglycoside inhibition of the 1α-subunit of the Na, K-ATPase. This in turn allows the design of de novo inhibitors of the 1α-subunit of the Na, K-ATPase. Several computer-based schemes using molecular modeling software may be employed to build potent and selective inhibitors from these scaffolds. Candidate scaffolds can be placed in the binding site of the target molecule, and a ligand can be built by successively bonding other fragments to it using methods known in the art. Thus, the Na, K-ATPase model and pharmacophores of the present invention will be used to guide the ‘growth’ of a potential inhibitor. Public and commercial chemical libraries (ACD, Maybridge, NCI, and WDI) may be searched for new molecules or scaffolds that satisfy the query's pharmacophore within specified tolerances. Further, computational tools will be employed to match small molecules with each other or with receptor topology rapidly based on similarity or complementarity in shape and electrostatic properties.

Promising candidate inhibitors emerging from the computer-aided design pipeline can be evaluated in terms of synthetic feasibility and overall acceptability. Following chemical synthesis, binding and inhibition assays known in the art can be used to evaluate the in vitro effects of the potential inhibitors.

Experimental Details

The following detailed examples are provided to illustrate the claimed invention better and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Sequence Alignment between SERCA1a and Na, K-ATPase

The sequences of the α1-subunits of the human, sheep, and rat Na⁺, K⁺-ATPases were obtained from GenBank (Accessions nos. P05023, P04074, P06685 respectively). The SERCA1a crystal structure (1EUL) was obtained from RCSB-PDB and the sequence was extracted using Insight II (Accelrys, San Diego, Calif.). The sequence alignment between SERCA1a and the α1-subunit of the sheep Na⁺, K⁺-ATPase was deduced from matrix based (PAM250 and Blosum35) approaches (www.ebi.ac.uk/clustalw/) and by threading the sheep sequence to the structural features of SERCA1a using THREADER (http://bioinf.cs.ucl.ac.uk) from University College London and the resulting alignment was further manually refined. The amino acid identity resulting from this alignment is 25.6% and all local areas of structural and functional importance are well conserved. The extracellular gaps resulting from the alignment are localized to loop regions (H1H2, and H7H8) and are thus easily accommodated.

Homology Modeling of the Na, K-ATPase

All molecular modeling operations were carried out using InsightII and related modules (Accelrys, Inc., San Diego, Calif.). The three-dimensional coordinates of the extracellular and transmembrane domains of the α1 subunit of sheep Na⁺,K⁺-ATPase were assigned. Undefined loops and gaps in the aligned sequences were accommodated by random generation of intervening sequences while conserving the integrity of backbone orientations in structurally defined, flanking residues. Inclusion of the randomly modeled regions required splice site relaxation in order to allow the protein backbone to adopt appropriate psi/phi angles. This operation was completed by relaxing the backbone atoms of those residues involved in splice sites and subjecting the molecule to a localized energy minimization procedure.

After generation of the model, the side chain residues were checked for acceptable rotomer positioning using the internal rotomer library and steric clashes were resolved by brief energy minimization of the entire molecule (250 iterations, steepest descent). Finally, the model was subjected to a full energy minimization through a series of independent steps in which hydrogen atoms, side chains, backbone atoms and, ultimately, the entire molecule were successively allowed freedom of movement. Similarly, the human and rat sequences were aligned to the sheep sequence, and then modeled and refined as described above. Point mutations as described herein were incorporated into the fully refined sheep structure, and the resulting mutants were again energy minimized through the steps discussed above. This procedure implemented the CVFF force field with a distance dependent dielectric function (ε=εₒr, with εₒ=4) until the convergence criterion of 0.04184 kJ/mol (0.01 kcal/mol) change in energy between successive iterations was achieved. The dielectric constant εₒ=4 was chosen as it corresponds to that of water in a fast-moving dynamic field.

Development of the Pharmacophore Model of the Na, K-ATPase

The 3D structure of each cardiotonic compound was built using the molecular fragment library of Sybyl6.6 (Tripos, Inc., St. Louis, Mo.) and based on the structure of digoxin as determined by X-ray crystallography.(ref) The conformation of the β- and γ-sugars was obtained by global energy minimization determinations. As previously described, the final structural models were energy minimized using the Tripos force field with the Gasteiger-Marsili method for assigning partial atomic charges.(ref) The GOLD operator usage probabilities for crossover, mutation, and migration were set to 95%, 95%, and 10%, respectively. The maximum distance between hydrogen bond donors and fitting points was set to 5 Å, and the nonbonded cutoff for the van der Waals energies was adjusted to the manufacturer recommended settings (10 kᵢⱼ=well depth of the van der Waals energy of the atom pair i,j). The ligand orientation accepted was the top scoring consensus orientation of 25 independent genetic algorithm (GA) runs, each with a maximum number of 1000 GA operations performed on a single population of 100 individuals. The selection pressure was 1.1. The centroid of the binding sphere was determined as the intersection of the vectors connecting H1H2 with H5H6, H1H2 with H9H10 and H5H6 with H9H10.

Binding energy calculations were performed as follows: ΔE_{binding} is defined as the difference between the potential energy of the ouabain-receptor complex (E_{complex}) and the sum of potential energies of the ouabain (E_{ligand}) and receptor (E_{receptor}).
ΔE_{binding}=E_{complex-(}E_{ligand}+E_{receptor})
Energy values represent the minimum potential energy resulting from the optimization procedure described above. Ouabain was allowed full freedom of movement throughout the process. Various orientations of ouabain with respect to the ATPase were obtained by combining the consensus orientations of ouabain as determined by the GOLD docking, with the appropriate homology modeled enzyme. Specifically, the bonded (Stretch, torsion etc.) and nonbonded (van der Waals and Columbic terms) energy terms were calculated by Discover (InsightII) whilst the linear Poisson-Boltzmann equation was solved using DelPhi to obtain the electrostatic contributions of the solvation energy (reaction field energy).
Design of Novel Na, K-ATPase Inhibitors

Candidate inhibitors identified from the receptor-based pharmacophores provided by the present invention will be further screened in terms of bioavailability and toxicological profile. Bioavailability is a measure of the value of the hydrophobic/hydrophilic balance, as measured by log P, will be predicted using the C log P program (Daylight Chemical Information Systems, Santa Fe, N. Mex.). Values of the aqueous solubility and cell membrane permeability will also be predicted using available molecular models in the laboratory as additional screens to select only the most promising candidate inhibitors. The toxicological profile of potential inhibitors will be determined by calculating relevant endpoints including carcinogenicity, Ames mutagenicity, developmental toxicity potential, skin irritancy, ocular irritancy, and aerobic biodegradability will be predicted for the candidate inhibitors using the QSAR-based TOPKAT program (Accelrys, Inc., San Diego, Calif.).

Several computer-based schemes may be employed to build potent and selective inhibitors from these scaffolds. First, a method known as “molecule growth” may be employed to place a ‘seed’ fragment or scaffold in the binding site of the target molecule, and to build a ligand by successively bonding other fragments to it using methods such as Small Molecule Growth (SmoG) (DeWhitte and Shakhnovich, J. Am. Chem. Soc. 118: 11733-11744, 1996)) or GrowMol (Bohacek and McMartin, J. Am. Chem. Soc. 116: 5560-5571, 1994). The Na, K-ATPase homology model of the present invention will be used to guide the ‘growth’ of a potential inhibitor.

Second, public and commercial chemical libraries (ACD, Maybridge, NCI, and WDI) may be searched using a fragment-based development method commonly known as “sub-structure searching.” This method uses a 3D pharmacophore as a query to search a chemical library for new molecules (or scaffolds) that satisfy the query's pharmacophore within specified tolerances. Third, a computational tool will be employed as a ray-tracing scheme to rapidly match small molecules with each other (or even with receptor pockets) based on similarity (or complementarity) in shape and electrostatic properties. It is a powerful and efficient method for drug discovery and particularly for the discovery of new scaffolds that share the same properties but not necessarily the same structural formula as the query molecule.

The design strategies used for chemical synthesis will depend on the structures of the proposed inhibitors. Promising candidate inhibitors emerging from the computer-aided design pipeline will be evaluated in terms of synthetic feasibility and overall acceptability. Following chemical synthesis, binding and inhibition assays will be used to evaluate the in vitro effects of the potential inhibitors. Assays will be conducted as describe previously (Biochemistry 2002, 41, 1137-1148). Briefly Na, K-ATPase will be purified from the outer medulla of frozen lamb kidney. Competitive binding assays will be performed in the presence and absence of varying concentrations of the potential inhibitors. Inhibition studies will be carried out with and without varying concentrations of the potential inhibitors, and the NADPH oxidation rate in the presence and absence of the inhibitors will be determined.

What is claimed is:

1. A novel pharmacophore model as defined by the parameters of Table 4 and Table 5.

2. The novel pharmacophore model of claim 1, wherein scaffold molecules derived therefrom can be used as a basis for compounds directed to inotropic Na, K-ATPase inhibition.

3. The novel pharmacophore model of claim 1, wherein the model produces an Na, K-ATPase inhibitor compound of the formula: 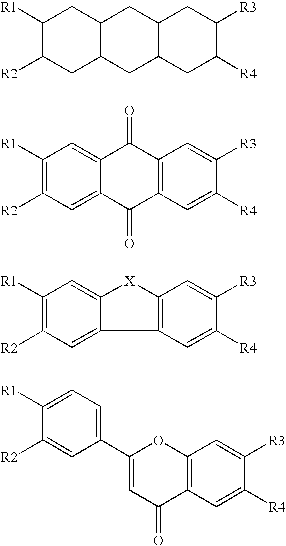
wherein R1, R2, R3 and R4 can be any organic functional group containing a hydrogen bond donor or a hydrogen bond acceptor and X is any element or group that allows the compound to retain inotropic activity.

4. The novel pharmacophore model of claim 3, wherein X is N, O, S, or C.

5. A method of using a pharmacophore model to create an Na, K-ATPase inhibitory compound comprising the steps of:
(a) creating alignment between SERCA and Na, K-ATPase, wherein SERCA is a template;
(b) transferring coordinates from the template to a model for structurally conserved regions;
(c) generating variable regions;
(d) refining the model through energy minimization steps; and
(e) performing docking analysis of prospective drug candidates.

6. The method of claim 5, further comprising the steps of:
(f) delineating the essential pharmacophoric elements for high binding affinity;
(g) searching databases of known compounds using the restraints as implicated by the pharmacophore with allowable tolerances; and
(h) utilizing de novo rational drug design and computer aided molecular modeling to design novel compounds using the restraints as implicated by the pharmacophore with allowable tolerances.

7. The method of claim 6, wherein the allowable tolerances in steps (g) and (h) is ±10%.

8. The method of claim 5, wherein step (a) is comprised of dynamic programming and threading.

9. The method of claim 5, wherein SERCA is SERCA1a.

10. The method of claim 5, wherein the steps are carried out using a computer-readable medium having computer-executable instructions.

11. The method of claim 10, wherein the steps are carried out using molecular modeling software.

12. A method of treating an individual with a heart disease comprising administering a therapeutically effective amount of an novel inotropic compound created using a novel pharmacophore model as defined by Table 4 and Table 5.

13. The method of claim 12, wherein the novel pharmacophore model produces novel inotropic drugs of the formula of:

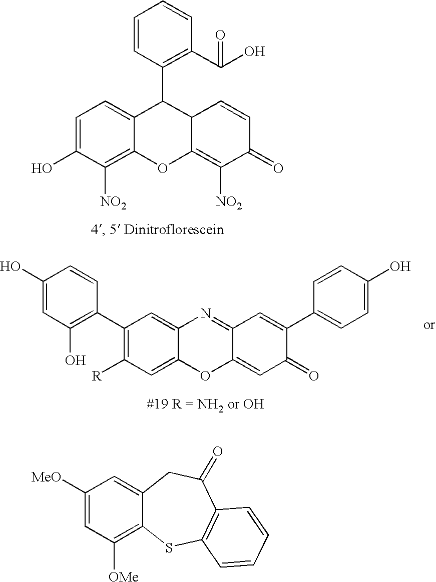

14. The method of claim 13, wherein the novel drugs have a wider therapeutic index than either ouabain or digoxin.

15. The method of claim 12, wherein the heart disease treated is congestive heart failure and supraventricular arrhythmia.

16. The method of claim 12, wherein the novel inotropic compound is administered in a pharmaceutically acceptable carrier.

17. The method of claim 12, wherein the novel inotropic compound is administered parenterally or orally.

18. The method of claim 12, wherein residues Q111, D121, E908 and M973 are unaltered.


SEQUENCE LISTING (2 sequences)

SEQ ID NO: 1 - protein, 1001 aa, Homo sapiens [sequence omitted]

SEQ ID NO: 2 - protein, 1016 aa, Ovis sp.
GRDKYEPAAVSEHGDKKKAKKERDMDELKKEVSMDDHKLSLDELHRKYGTDLNRGLTTARAAEILARDGPNALTPPPTTPEWVKFCRQLFGGFSMLLWIGAVLCFLAYGIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQDNLPILKRAVAGDASESALLKCIEVCCGSVKEMRERYAKIVEIPFNSTNKYQLSIHKNANAGEPRHLLVMKGAPERILDRCSSILIHGKEQPLDEELKDAFQNAYLELGGLGERVLGFCHLMLPDEQFPEGFQFDTDDVNFPVDNLCFVGLISMIDPPRAAVPDAVGKCRSAGIKVIMVTGDHPITAKAIAKGVGIISEGNETVEDIAARLNIPVSQVNPRDARACVVHGSDLKDMTPEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPQTDKLVNERLISMAYGQIGMIQALGGFFTYFVIMAENGFLPNHLLGIRVTWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY